(19) United States
(12) Patent Application Publication
(10) Pub. No.: US 2014/0275851 A1
(43) Pub. Date: Sep. 18, 2014

(54) MULTI-SITE DATA SHARING PLATFORM

(71) Applicant: eagleyemed, Inc., Santa Clara, CA (US)

(72) Inventors: Ravi N. AMBLE, San Jose, CA (US); Harish P. HIRIYANNAIAH, San Jose, CA (US); Farooq Mirza Mohammad RAZA, Milpitas, CA (US); Steven J. SALVE, San Jose, CA (US)

(21) Appl. No.: 14/292,258

(22) Filed: May 30, 2014

Related U.S. Application Data
(63) Continuation-in-part of application No. 14/214,321, filed on Mar. 14, 2014.
(60) Provisional application No. 61/800,316, filed on Mar. 15, 2013.
(60) Provisional application No. 61/829,905, filed on May 31, 2013.

(51) Int. Cl. A61B 5/00; A61B 6/03; A61B 8/00; A61B 5/145; A61B 5/0402; A61B 5/0476; A61B 5/1455; A61B 5/01; A61B 5/055; A61B 5/0205

(52) U.S. Cl. CPC A61B 5/0013; A61B 5/0077; A61B 5/055; A61B 6/032; A61B 5/0035; A61B 5/7425; A61B 5/743; A61B 5/7475; A61B 8/463; A61B 8/565; A61B 5/02055; A61B 5/0402; A61B 5/0476; A61B 5/14551; A61B 5/01; A61B 5/14532; A61B 5/02416
USPC 600/301; 600/476; 600/411; 600/427; 600/440; 600/509; 600/544; 600/324; 600/549; 600/365; 600/407; 600/300; 600/529; 600/485; 600/483; 600/484; 600/323

(57) ABSTRACT

A variety of methods and arrangements for sharing medical data are described. In one aspect, one or more data streams are received from one or more medical imaging/sensing or other types of devices. Frames are obtained from the streams. In some embodiments, particular frames and/or parts of frames are selectively encrypted. The frames are transmitted to a remote device, where they are rendered and/or displayed at the remote device. In various embodiments, the frames of different streams are synchronized.

### RELATED APPLICATIONS

The present application is a Continuation-in-Part of earlier filed U.S. patent application Ser. No. 14/214,321 filed Mar. 14, 2014 which claims priority of U.S. Provisional Patent Application No. 61/800,316 filed Mar. 15, 2013. The present application also claims priority of U.S. Provisional Application No. 61/829,905 filed May 31, 2013. Each of these priority applications is incorporated by reference in their entirety for all purposes.

### FIELD OF THE INVENTION

The present invention relates generally to medical technologies. More specifically, the present invention involves a telemedicine platform that allows medical professionals to share data, images and video.

### BACKGROUND

In the medical field, different medical procedures and examinations require varying levels of expertise. Some examinations and/or procedures can be conducted by a nurse practitioner, while others are typically done by a medical doctor (or a veterinarian in the case of animal medicine), while still others require the participation of a highly trained medical specialist. One very specialized medical field is radiology. Radiology is a medical specialty that employs the use of imaging to diagnose and/or treat disease or trauma within the body. Radiologists use an array of imaging technologies including ultrasound, X-ray radiography, computed tomography (CT), nuclear medicine, positron emission tomography (PET) and magnetic resonance imaging (MRI) to diagnose or treat ailments.

In human medicine, the technician who conducts certain types of medical tests often lacks the expertise to properly interpret the results. As a result, a specialist is contacted to provide a diagnosis based on the results of the examination. For example, the acquisition of internal medical images is usually carried out by a radiographer or a radiologic technician in a radiology lab without a radiologist or the ordering physician being present. Due to the complexity of radiological images, even the surgeons and primary care physicians who order the radiologic examination typically cannot independently make a diagnosis based on the radiological images. Rather, a certified radiologist must interpret or “read” the images and produce a report of their diagnosis, findings and/or impressions. Since the radiologist is most often not present during the radiology session, the images must be sent to the radiologist for analysis after the session has been completed. Once the radiologist has completed their analysis, a report is typically transmitted to the ordering physician—who schedules an even later appointment to meet with the patient to discuss the results. If the radiologist sees something that requires further imaging (e.g., to get a different view of a region of interest)—a new scan is ordered and the process is repeated. This substantially increases the time and costs involved in obtaining a diagnosis of a medical condition

The process can be sped-up if the radiologist personally conducts the radiological examination or is present during such examination. However, there are a limited number of radiologists and it is often not practical from either a cost or availability standpoint for the radiologist to be physically present during the radiological examination. Therefore, the most common process is to acquire the radiological images in a first session and then transmit the images to the radiologist for review after the radiology session is complete. This problem is amplified in the veterinarian medicine field where there are only a few hundred radiologists that collectively service the needs of tens of thousands of veterinary clinics.

More generally, the need to obtain the assistance of a specialist can introduce substantial delay in the diagnosis of a variety of different types of medical conditions. In some fields of medicine and in some parts of the country, there are a very limited number of specialists available. As a result, in can take weeks (or even months) to get appointments with the appropriate specialist(s) and arrange for the required lab work (e.g., radiologic imaging, EKG, etc.). It is not uncommon for a specialist, reviewing test results to request another test or scan. This may be because the test was improperly performed. Alternatively, the results may indicate to the specialist that the test should be performed in a somewhat different manner or that further tests may be appropriate. As a result, both the diagnostic exam/test and the consultation with the specialist must be rescheduled.

Telemedicine has the potential to substantially improve patient care by facilitating more immediate access to highly trained specialists at a variety of stages in the health care process. Accordingly systems that can improve the efficacy of remote medicine are of great interest.

The Applicant has developed a collaborative telemedicine platform that allows a remote medical specialist (such as a radiologist) to participate in an examination in real time. Several unique aspects of that platform are described herein. Although radiological applications are often used as a representative use of the technology, it should be apparent from the following description that the described collaborative telemedicine platform can also be used in a wide variety of other remote medicine applications.

Radiologists and other specialists sometimes like to refer to the cases studies, professional references and/or the medical literature to identify similar cases when making a diagnosis. Therefore, there are continuing efforts to provide more useful on-line tools to make such resources readily available and/or to make it easier to access the desired information. In radiology, it can be helpful to find similar radiologic images to help make, or confirm a diagnosis.

### SUMMARY

A variety of methods and arrangements for sharing medical data are described. In one aspect, multiple data streams are received from one or more medical imaging/sensing devices or other types of devices (e.g., a video camera). Frames are obtained from the data streams. In some embodiments, a part of each frame and/or only particular frames are selectively encrypted. The frames are transmitted to a remote device. The frames for the streams are reconstructed, rendered and/or displayed at the remote device. In various embodiments, the frames of different streams are synchronized.

The streams may involve a variety of different types of media and data, depending on the needs of a particular application. In some embodiments, for example, the streams are a video stream and a biometric imaging stream. One example approach involves performing a biometric imaging scan (e.g., an ultrasound scan) on a patient, which generates a biometric image stream. Concurrently, a video camera is directed at a technician that is using the biometric imaging device, which indicates how the device is being handled and positioned. The video camera generates a video stream. Frames are obtained from the video and biometric imaging streams. The frames are transmitted to a remote device e.g., as a packet sequence. At the remote device, the frames are reconstructed and synchronized. In various embodiments, a user of the remote device can then display and observe the video and biometric imaging in (near) real time. The synchronization helps ensure that the video and biometric imaging are properly timed and coordinated when viewed at the remote device.

Any suitable data streams may be synchronized. In some embodiments, for example, frames are obtained from biometric waveform data and biometric imaging streams received from one or more medical imaging/scanning devices. The frames are transmitted and synchronized. In still other embodiments, annotation data is received from a specialist or medical professional and biometric imaging data is received from a medical imaging/sensing device. Frames are obtained from the annotation data and the biometric imaging data, which is then transmitted and synchronized.

In another aspect, a data stream is received from a medical imaging/sensing device or another type of device. The data stream may be any suitable type of data stream, including a biometric imaging, biometric data, video, audio, annotation data or other type of data stream. Frames are obtained from the stream. At least some of the frames are (partially) encrypted. In some embodiments, only a part of each frame is encrypted. Some implementations involve encrypting only a header of the frame, at least part of a header of a frame or only part of the header and part of the media data (payload) of the frame. The frames are then transmitted to a remote device.

### BRIEF DESCRIPTION OF THE DRAWINGS

The invention and the advantages thereof, may best be understood by reference to the following description taken in conjunction with the accompanying drawings in which:

FIG. 1 is a block diagram of a multi-site data sharing platform according to a particular embodiment of the present invention.

FIG. 2 is a block diagram of a patient telemedicine system according to a particular embodiment of the present invention.

FIG. 3 is a block diagram of a specialist telemedicine device according to a particular embodiment of the present invention.

FIG. 4 is an example user interface for the patient telemedicine device illustrated in FIG. 2.

FIG. 5 is an example user interface for the specialist telemedicine device illustrated in FIG. 3.

FIGS. 6 and 7 are flow diagrams illustrating a method for receiving, encoding, encrypting and transmitting data streams according to a particular embodiment of the present invention.

FIG. 8 is a data encoding module according to a particular embodiment of the present invention.

FIGS. 9A-9D are block diagrams illustrating possible encryption schemes for frames according to various embodiments of the present invention.

In the drawings, like reference numerals are sometimes used to designate like structural elements. It should also be appreciated that the depictions in the figures are diagrammatic and not to scale.

### DETAILED DESCRIPTION

The present invention relates generally to methods and arrangements for supporting collaborative telemedicine. The Applicant has developed a collaborative telemedicine platform that allows a remote medical practitioner (who may be a specialist such as a radiologist, a general practitioner, etc.) to participate in an examination in (near) real time. Several unique aspects of that platform are described herein.

In one aspect, a platform is described that allows a practitioner conducting a medical examination and a remote medical practitioner to concurrently share a plurality of different views in real time. By way of example, one of the shared views may be live streamed biometric information (such as radiological images, biometric waveforms, etc.). Another shared view may show a relevant view of the patient being examined. One example of this might be a view showing the placement and orientation of an ultrasonic probe being used in a sonographic examination. Other shared views may include items such as video conference type view of one of the participants, a replay of an imaging stream selected by one of the participants, reference materials that have been selected by one of the participants, etc. Several unique aspects of that platform are described herein. A particular strength of the platform is the ability to share medical imaging streams (such as the output of an ultrasonic probe) with remote collaborators in real time as the examination is taking place. Although radiological applications are often used as a representative use of the technology, it should be apparent from the following description that the described collaborative telemedicine platform can also be used in a wide variety of other remote medicine applications as well.

Referring initially to FIG. 1, a multi-site data sharing platform 100 according to a particular embodiment of the present invention will be described. The platform 100 includes a patient telemedicine system 102, which includes a first telemedicine device (workstation) 104. The platform 100 further includes second and third telemedicine devices (workstations) 108 and 110. For convenience, the first telemedicine device 104 is sometimes referred to herein as the “patient” or “local” telemedicine system since it is preferably positioned at the location of a patient and is used by a technician or practitioner that is interacting with the patient. The second telemedicine device 108 is sometimes referred to herein as the “remote” or “specialist” telemedicine device since it is typically positioned at a location that is apart from the patient and is most often used by a medical practitioner (such as a specialist, the ordering doctor, etc.) that is participating in the telemedicine session. Optionally, one or more additional remote telemedicine devices 110 may be included for use by others that are participating in or viewing the telemedicine session. Such remote participants may include other specialists that are participating to provide a second opinion, other medical practitioners involved in the patient's care (e.g., the ordering physician/veterinarian, a surgeon that will be operating on the patient, the patient's primary care physician, etc.), parties that are observing the examination for training or educational reasons (e.g., interns, residents, students trainees, etc.) or anyone else that has a reason to participate in the examination.

The patient telemedicine system 102 and the telemedicine devices 104, 108 and 110 are connected to one another through one or more networks 112 and may optionally also be connected to a cloud based architecture 114. The cloud based architecture 114 preferably includes a server 116 and a database 118 which optionally include a medical records store and various other databases. Any suitable network(s) may be used to connect the devices of the platform, including but not limited to local area networks, wide area networks, intranets, the Internet, etc.

The patient telemedicine system 102 includes one or more medical imaging/sensing devices 106 and a patient telemedicine device 104. The telemedicine devices 104 typically take the form of a general purpose computing device having software configured to perform the described functions—although special purpose devices can be used as well. Suitable computing devices include desktop computers, laptop computers, tablet computing devices, mobile devices, etc.

The patient telemedicine device 104 is arranged to obtain (and optionally store) data from each connected medical imaging/sensing device that is being used for diagnostic testing. In many circumstances, the data received from a particular source will be received in the form of one or more live streams (e.g. a sonographic stream, or a multiplicity of sensor outputs from an EKG machine). Generally, the patient telemedicine system 102 is situated near a patient who is currently undergoing the diagnostic testing, although this is not a requirement. The patient telemedicine device 104 encodes the data streams from the diagnostic testing and transmits them to other participating telemedicine devices (e.g., specialist telemedicine device 108). As a result, users of those remote telemedicine devices are able to view and participate in the diagnostic test in (near) real time. That is, the media received at the remote telemedicine devices is generally rendered live, although there may be some small delay due to the inherent latencies caused by network conditions and overhead. The patient telemedicine system 102 also allows a medical professional at the site of the diagnostic test to communicate with professionals who are participating in the telemedicine session remotely.

The patient telemedicine device 104 is preferably arranged so that it may be coupled to (or otherwise receive inputs from) a variety of different types of biometric diagnostics machines. These may include various types of imaging devices (e.g., ultrasound probes, X-ray machines, MRI devices, CT scanner, etc.) and/or biometric measurement devices (e.g., EKG, EEG, or ECG devices, pulse oximeters, thermal/temperature sensors, blood pressure monitors, glucose level monitors, pulmonary function testers, etc.).

The patient telemedicine device 104 is also arranged to provide an audio and one or more video links with the remote telemedicine devices. The audio link allows the practitioner(s) who are working with the patient to talk with the remote participants. The video links originating from the patient side allow remote participants to view relevant aspects of the examination. In various embodiments, a video camera is attached to the device 104 to provide video of the procedure that may be shared with the remote participants. The appropriate focal point of the camera will vary with the needs of any particular examination. Most often, a camera will be focused on the examination target. For example, during an ultra-sound examination, a camera may be focused on the area that the ultrasound probe is being applied to so that remote participants can see how the operator is positioning and otherwise using the probe simultaneously with viewing the images produced by the probe. In other situations the telemedicine device 104 may be arranged to receive a video feed from a camera associated with an endoscope during an endoscopic procedure. Sharing such streams allows the remote participants to see the video output of the endoscope.

There are times when the operator of the patient telemedicine system 102 will want to view various types of supplementary information that may be helpful in the diagnostic/medical procedure. To facilitate this, the system has access to use cases, medical records, reference imagery and other types of data that may be helpful in the diagnostic/medical procedure. In various embodiments, such data is downloaded through the cloud based architecture 114 from the server 116 and/or the database 118 to the patient telemedicine system 102.

Depending on the preferences of the operator of the patient telemedicine system 102, some or all of the above data is selectively presented in a display that is connected to or part of the system 102. Preferably, the patient telemedicine device 104 has a graphical user interface that allows the operator to select and simultaneously display a plurality of different items as different views. The different views may be rendered in different windows, in different panes of a window, or in any other suitable manner. As will be described in more detail below, the operator may select the views that are deemed appropriate at any time. By way of example, when used in conjunction with an ultrasound imaging machine, one window may display the ultrasound image live. A second window may show the output of video camera 106a and a third window may display a video conference feed from a remote participant. A fourth window may be arranged to show a patient's medical record or other information of use to the operator. An advantage of locally displaying the locally generated biometric and video feeds is that it allows the operator to see what is being shared with remote participants. In another example, the operator can arrange to have biometric images (e.g., an ultrasound scan displayed in real time) presented in one window of a display screen and biometric data (e.g., waveform results from an EEG) in another window. A video of a remote collaborator might be presented in a third window, and a fourth window can include a medical record and/or reference imagery (e.g., if the ultrasound scan shows a diseased heart, the fourth window may include a reference image of a healthy heart.)

In various embodiments, an operator of the patient telemedicine system 102 is able to annotate any of the displayed images, waveforms or test results. In some implementations, for example, the operator can provide input (e.g., circle or otherwise mark a portion of an image displayed on a touch-sensitive screen) indicating that a particular image part should be highlighted or examined further. This annotation data is also selectively transmitted to other remote collaborators in the platform.

The operator can selectively designate which of the above types of data should be transmitted or shared with other remote collaborators in the multi-site data sharing platform 100. The patient telemedicine device 104 obtains or generates frames from the shared data, selectively encrypts, multiplexes and/or transmits them through the network to the other collaborator(s) and telemedicine device(s).

Each remote telemedicine device 108/110 receives the frames and is arranged to render them in real time. The various media streams may be shown on one or more display screens at the remote telemedicine device. The operator of each remote telemedicine device 108/110 can configure how the various types of media are displayed. For example, if the patient telemedicine device transmits biometric imaging data, waveform data, video data, an audio stream and patient records to the remote telemedicine device 108/110, the operator of the remote telemedicine device can display the waveform data in one window on the display screen, the biometric imaging data in another window, the video data in a third window, and the patient records in a fourth window. Received audio communications will be played through a speaker associated with the specialist telemedicine device 104. Generally, the various media streams are received simultaneously, in real time, and rendered at nearly the same time and order as they are received or displayed at the patient telemedicine device 104.

The operator of the remote telemedicine device 108/110 can also provide input to the device, which is transmitted in real time to other collaborators in the platform 100. By way of example, the operator can annotate any received biometric image, waveform or test result or select a relevant segment of a stream for review (e.g., create a short video segment or “cine-loop”) and any of these items can be shared with other collaborators. (Stream segments that are typically rendered in the form of a short video sequence that is repeatedly replayed are sometimes referred to herein as “cine-loops.”) The operator can also speak with other participants over an audio channel. The operator can also obtain use cases, medical records, reference imagery and any other suitable diagnostic aids or information from the cloud-based server 116 and/or the database 118. When appropriate, the operator can elect to share any of these inputs with other participants in the telemedicine session. Shared media (i.e., annotation data, medical records, use cases, audio messages, etc.) are then transmitted to the patient telemedicine device 104 and/or any other designated devices in the platform, so that they can be rendered and displayed in real time at those devices.

The above system allows a specialist to use the device 108/110 to fully participate in the aforementioned diagnostic procedure. In various embodiments, for example, the specialist telemedicine device 108/110 receives ultrasound imagery. Simultaneously, the specialist also receives a video indicating how an ultrasound probe is being handled to produce the ultrasound imagery. The specialist is thus able to review the procedure in real time. Thus, the specialist has the ability to provide feedback to the medical professional who is handling the ultrasound probe or equipment. For example, the specialist can request that the attending medical professional reposition an ultrasound probe on a particular portion of the patient's body to get a better or different view of a region of interest.

The server 116 is arranged to facilitate communication between participants in a telemedicine session. In some embodiments, particularly in implementations in which there are only two participants (e.g., the patient telemedicine device 104 and a single specialist telemedicine device 108), the server 116 is not necessary. In other applications, however, there may be more than two participants. For example, there may be a second specialist who is using another specialist telemedicine device 110 to also participate in and observe the diagnostic procedure. In some embodiments, some or all traffic between multiple telemedicine devices passes through the server. The server 116 helps ensure that each participating device has access to any data that is shared by any other device.

The server 116 can provide a variety of additional features, depending on the needs of a particular application. Various implementations involve the server 116 providing a store and forward and broadcasting functionality. That is, any data that is to be shared and is transmitted from a device is first transmitted to the server, which stores the data and then forwards copies of the data to the intended recipients. In some embodiments, the server 116 and/or its connected database(s) 118 store copies of some or all traffic transmitted between the devices in the platform. Upon request from any properly authorized device (e.g., patient telemedicine device 104 and specialist telemedicine device 108/110), the server 116 and/or the database 118 can provide previously transmitted imagery, test results, annotations or any other transmitted data which can be a powerful diagnostics tool.

Referring next to FIG. 2, a representative embodiment of the patient telemedicine system 102 of FIG. 1 will be described. The patient telemedicine system 102 is arranged to collect data streams from various medical imaging/sensing devices 106a-106e that are being used on a patient, process the streams, and transmit them to designated recipients, such as a specialist. In the illustrated embodiment, the patient telemedicine system 102 has the ability to received inputs from a variety of different devices such as a video camera 106a, a probe 106b, a probe platform 106e, an image acquisition device 202, an echocardiogram (EKG) device 106c, an X-ray device 106d and a display device 210. It should be noted that the figure is intended to be illustrative and exemplary and that any number or combination of components, medical imaging/sending devices and tools may be used. All of these devices are connected with and transmit data to the patient telemedicine device 104.

The video camera 106a represents one or more video cameras used to take video footage of an area of interest, a medical scanning/sensing device, a patient, a technician and/or an operator of the telemedicine device 104. In various embodiments, for example, the video camera 106a is directed at a medical scanning/sensing device (e.g., an ultrasound probe) and/or a particular region (e.g., the hand of a technician that is gripping the probe and/or a portion of the patient's body where the probe is being applied, such as the chest or abdominal area.) Thus, the video footage can be used to observe how the medical scanning/sensing device or probe is being applied to a patient, and a viewer of the footage can make recommendations on how the device should be used or repositioned. Alternatively or additionally, a video camera 106a is directed at an operator, medical professional or other person who is participating in the telemedicine session from the side of the patient. A video camera 106a may also be taking video footage of the patient. The video data is streamed to the patient telemedicine device 104 in (near) real time.

The probe 106b represents any type of medical imaging/sensing device that is operated and handled by a medical professional. An example of such a probe is an ultrasound probe, which is a wand or other device that emanates ultrasound waves and is typically placed against the body of a patient to provide a scan of a particular portion of the patient's body. The probe 106b is attached to a probe platform 106e, which is arranged to collect the image data. An image acquisition device 202 is attached to the probe platform 106e and is arranged to obtain the biometric image stream generated by the probe platform 106e. The image acquisition device 202 is in turn connected to the patient telemedicine device 104 and transfers the biometric image stream to the telemedicine device 104, so that it can be encoded and transmitted, as desired, to a remote telemedicine device (e.g., specialist telemedicine device 108/110) and other participants in the telemedicine session.

The electrocardiogram device 106c is arranged to monitor the electrical activity of the heart of the patient. In various embodiments, an electrocardiogram involves attaching multiple electrodes to the body of the patient. The electrodes monitor the electrical activity in the body and an electrocardiogram device generates waveforms to indicate this electrical activity. The electrocardiogram device transmits this waveform data to the telemedicine device 104. The electrocardiogram device 106c may represent any number of suitable devices that are arranged to collect biometric data that tracks or reflects physiological changes in the patient. Such devices include but are not limited to an electroencephalography (EEG) device, a temperature detection device, a blood pressure device, a glucose level detector, a weighing device, a pulmonary function test device and a pulse oximeter.

The X-ray device 106d is arranged to project X-rays towards a patient and obtain X-ray images of portions of the patient's body. Any device suitable for obtaining biometric image data may be added to or replace the X-ray device. Such devices include but are not limited to a CT scanner, an MRI device, a retinal scanner, an ultrasound scanning device or any nuclear medicine-related device. The X-ray device generates biometric imaging data, which is also transmitted to the telemedicine device 104.

The patient telemedicine device 104 coordinates the operation of the other components of the patient telemedicine system 102. The patient telemedicine device 104 collects data from multiple sources and medical imaging/sensing devices, displays the data, makes requests for additional data, receives input from an operator and shares selected types of data with other participants in a telemedicine session. Any suitable type of computing device may be used. In some embodiments, for example, the patient telemedicine device 104 is a laptop, computer tablet, a mobile device and/or a computer.

In addition to the imaging, waveform and other types of medical data described above, the patient telemedicine device 104 can also receive a variety of other types of data from the cloud based architecture 114 and/or from an operator of the device 104. In various embodiments, there is an audio channel or messaging system that allows an operator to communicate with other participants in the telemedicine session by speaking into a microphone 208 that is connected to the patient telemedicine device 104. Additionally, the operator can provide input to the telemedicine device 104 to request supplementary data 206. The telemedicine data then transmits a request for such data to the cloud-based server 116 and/or database 118. The supplementary data is any suitable diagnostic or reference data that will assist in the diagnostic procedure. The supplementary data 206 includes but is not limited to use cases, reference imagery (e.g., ultrasound images of healthy or unhealthy tissues in the body, etc.), medical records for the patient, descriptions of various medical diseases and conditions, etc. Upon request, the server 116 transmits the requested supplementary data to the patient telemedicine device 104.

The patient telemedicine device 104 may also receive collaboration data 204 from other devices in the same telemedicine session (e.g., specialist telemedicine device 108/110 of FIG. 1.) The collaboration data 204 includes any suitable data that a remote specialist or operator chooses to share with the rest of the participants in the telemedicine session, including but not limited to annotations, audio messages, selected use cases, reference imagery and medical records.

The patient telemedicine system 102 includes a display device 210, which is part of or connected to the patient telemedicine device 104 and may be any suitable video screen or user interface suitable for presenting test results and media. The operator of the telemedicine device 104 can select which, if any, of the above types of data should be displayed at the display device 210.

One example user interface 400 displayed in the display device 210 is illustrated in FIG. 4. The user interface 400 includes multiple windows, including windows 402, 404, 406, 408 and 410. In various embodiments, the operator of the telemedicine system 104 is able to configure the user interface 400 in accordance with his or her preferences. For example, some of the received imaging, waveform, supplementary or collaboration data may be excluded from the user interface, while other selected media is shown. Each type of media can be shown in a separate window, which can be resized and moved as desired by the operator.

In the illustrated embodiment, for example, an image from an ultrasonic scan, taken using the probe 106b, is displayed in the window 402. In this case, the image is a snapshot from an ongoing ultrasound scan of a dog. The image is constantly received and updated in (near) real time as the scan continues. The medical records for the pet, which was downloaded as supplementary data from the cloud-based server, is presented in window 406. A (near) real time video of a technician performing the ultrasonic scan is shown in window 410. This video was obtained from a video stream generated by the video camera 106a. Also, in window 408, a real time video of a specialist using the specialist telemedicine device 108 is shown. This video is collaboration data that was transmitted by the specialist telemedicine device 108 for display at the patient telemedicine system 102.

In another window 404, various reference ultrasound images are presented that are used to provide a comparative model for the ultrasound imagery in window 402. In some embodiments, the patient telemedicine device 104 requests and receives such images from the cloud-based server 116 or database 114. In other embodiments, the patient telemedicine device 104 receives a message from a specialist (i.e., through a specialist telemedicine device 108/110), which identifies imagery or other data that is stored in the cloud and should be reviewed. In response to the message, the user can provide input to the patient telemedicine device 104, which triggers the downloading of the images from the cloud-based database 118 for display at the display device 210.

In this example, the media in windows 402, 408 and 410 are received in real time from various connected medical imagery/sensing devices (e.g., the probe, the video camera) or from a specialist telemedicine device 108/110. Additionally, any suitable type of collaboration data received from a specialist is generally also received and displayed in real time. For example, the video of the specialist in window 408 can display, in real time, the face of the specialist, since face-to-face conversations are sometimes desirable and can facilitate communications between the participants in the telemedicine session. This video is constantly and progressively transmitted by the specialist telemedicine device 108 to the patient telemedicine device 104 over the network.

Some implementations allow an operator to annotate any of the images displayed in the user interface. In various embodiments, the operator is able to mark, circle or highlight any of the windows. The operator can provide such marks by, for example, touching or swiping a touch sensitive screen, applying a pen to the screen, or by providing keyboard or other types of input. The patient telemedicine device 104 receives the annotation data 212 and associates it with the frames, data or images upon which the marks were made. This information can then be transmitted in real time to other devices in the multi-site data sharing platform (e.g., the specialist telemedicine device), so that they may review the markings as they are made.

The operator of the patient telemedicine device 104 can then provide input to the device 104, designating which types of the aforementioned data (e.g., biometric imaging data, annotation, video, audio, etc.) should be shared with one or more other devices in the platform. Alternatively, this sharing selection process may be automated, possibly based on settings that were configured by the operator beforehand. The patient telemedicine device 104 receives the designated data streams and selectively encrypts frames or parts of frames in the data streams. In various embodiments, the frames are broken down into packet sequences 214, which are multiplexed and/or transmitted to a device 108/110 and/or to the server 116 for distribution throughout the multi-site data sharing platform 100. The frames are then reconstructed at those remote devices for rendering at the devices.

Referring next to FIG. 3, the specialist telemedicine device 108 illustrated in FIG. 1 will be described. The specialist telemedicine device includes or is connected to a video camera 110, a microphone 302 and a display device 312. In the illustrated embodiment, the specialist telemedicine device 108 is used by a specialist (e.g., a radiologist) whose expertise is desired by a user of the patient telemedicine device 104. In some situations, another medical practitioner (e.g., a family doctor for the patient) uses the specialist telemedicine 108 to remotely observe the diagnostic procedure. The specialist telemedicine device 108 may be any suitable computing device, including but not limited to a computer, a laptop, a computer tablet and a mobile device.

The specialist telemedicine device 108 allows its operator to view biometric and medical data in (near) real time and participate in a diagnostic procedure that is being currently performed on a patient at the site of the patient telemedicine system 102. The specialist telemedicine device 108 includes a network interface that receives the aforementioned packet sequences sent from the patient telemedicine device 104 (e.g., biometric imaging data 318, biometric data 320, video 324, audio 322, collaboration data 326, supplemental data 316, annotations 314, etc.) Additionally, any authorized device in the multi-site data sharing platform 100 can selectively transmit data for review and rendering to the specialist telemedicine device 108.

The specialist telemedicine device 108 receives the packet sequences 214 and reconstructs the frames of the data (e.g., biometric imaging data, annotation, video, audio, etc.) The frames can then be rendered and the various types of data can be displayed. The operator of the specialist telemedicine device 108 can provide input to the device, indicating which types of media and data should be displayed at the display device 312. The display device 312 may use any suitable user interface, screen or display technology, including but not limited to a video screen, a touch sensitive screen and an e-ink display. In various implementations, different types of data are shown in separate windows or sections on a user interface 500 displayed on the display device 312.

An example user interface 500 is illustrated in FIG. 5. The user interface 500 includes multiple windows 502, 504, 506, 508 and 510. Different types of media are shown in separate windows. By way of example, an ultrasound image is shown in windows 502. A video of an ultrasonic probe being applied to the body of a patient is shown in window 508. A video of the specialist at the specialist telemedicine system 108 is shown in window 510. The operator of the specialist telemedicine device 108 may configure, resize, move and select media for each window as described in connection with the user interface FIG. 4. In some embodiments, the operator can remotely control the camera generating the video in window 508, allowing the operator to zoom in or out or focus the video camera at different areas of interest, which then adjusts the video in window 508 accordingly and in (near) real time.

It is assumed that the user interface 500 of FIG. 5 is being presented at approximately the same time that the user interface 400 of FIG. 4 is being presented at the patient telemedicine system. As previously discussed, in the example illustrated in FIG. 4, the display device 210 for the patient telemedicine system 102 is displaying an ultrasound image and a video of an ultrasound scanning procedure in (near) real time as the image is being generated at the probe 106b and as the ultrasonic scan is being performed. These biometric imaging and video streams have been transmitted to the specialist telemedicine device 108, so that they may be displayed (nearly) simultaneously in windows 502 and 508 of the user interface 500. Thus, an operator of the specialist telemedicine device is able to observe the biometric images and the handling of the biometric imaging equipment at the patient telemedicine system 102 in (near) real time.

Windows 504 and 506 display the supplementary data 316 (reference images and medical records) described in connection with the user interface 400 of FIG. 4. In some situations, the specialist using the specialist telemedicine device 108 initially requests the supplementary data. In that case, the specialist provides input to the specialist telemedicine device 108, which causes the device 108 to send a request for the selected data to the cloud-based server 116 and/or database 118. The desired supplementary data is then downloaded into the device 108 and presented in a user selected window of the user interface 500. Alternatively, another professional at a remote device (e.g., patent telemedicine device 104) may have been the first to suggest the use of the supplementary data. In that case, the remote device (e.g., the patient telemedicine device 104) transmits a message to the specialist telemedicine device 108 recommending particular types of supplementary data which are available in the cloud-based architecture 114. In response, the operator of the specialist telemedicine device 108 provides input to the device 108, which causes the device 108 to retrieve the suggested data and display it at the display device 312.

It should be appreciated that all of the packet sequences for the various types of data (e.g., biometric imaging data, biometric data, waveform data, video, audio, etc.) are received simultaneously and optionally rendered and displayed in (near) real time at the display device 312. This allows the specialist to review the images as they are being generated (e.g., at the patient telemedicine system) and/or follow the diagnostic procedure as it is taking place. Additionally, audio messages from any other device in the platform 100 are played over a speaker, allowing the specialist to listen to commentary from other participants in the telemedicine session.

The specialist telemedicine device 108 allows the specialist to create and share audio messages, make annotations, obtain and suggest supplementary data. Generally, such operations are handled in a similar or the same manner as with the patient telemedicine device 104 of FIG. 2. That is, the operator of the specialist telemedicine device 108 can mark or annotate any displayed images or information, recommend use cases, reference images or other types of supplementary data, and create audio messages using the microphone 302. Additionally, the telemedicine device 108 can include a video camera 310, which can take video footage of the specialist or any other suitable target. The operator of the specialist telemedicine device 108 can provide input to the device, identifying which of the above data that should be shared. The operator can further provide input to the device 108 indicating which devices (e.g., another specialist telemedicine device 110, the patient telemedicine device 104, etc.) in the telemedicine session should receive the selected data. (In various embodiments, the operator makes such selections prior to the beginning of the telemedicine session. During the session, different types of data are then automatically shared and transmitted based on the selections.) The selected data (e.g., annotations, recommended supplementary data, etc.) is progressively transmitted as it is created to the designated recipient devices, so that it can be selectively displayed in (near) real time at those devices.

Referring next to FIGS. 6-7, a method 600 for receiving, encoding and transmitting data streams according to a particular embodiment of the present invention will be described. Generally, the method 600 is performed where diagnostic testing is taking place i.e., at the patient telemedicine system 102 of FIG. 2. When multiple data streams are received from various medical imaging/sensing devices, they can be separately encoded, compressed and/or encrypted based on their individual characteristics. Selected streams can also be synchronized, so that telemedicine participants at remote devices can view multiple images, waveforms and video in the appropriate order and in (near) real time.

At steps 602, 604, 606 and 608, the patient telemedicine device receives one or more types of data streams simultaneously from various imaging/sensing/video devices. For example, a video camera 106a takes video footage of an area of interest (e.g., a part of the body where an ultrasound probe is being applied) and streams the footage to the device (step 602). A suitable medical sensing device (e.g., an EKG 106c, a heart monitor, a blood pressure monitor, etc.) monitors a medical condition of a patient and transmits biometric waveform data to the device 104 (step 604). In various embodiments, a medical imaging device (e.g., an ultrasound scanner and probe 106b) collects images from the patient and sends them to the device 104 (step 606).

Any combination of data streams may be received at the device. By way of example, one useful combination involves an ultrasound probe and a live video camera feed. The video camera that provides the feed is directed at an ultrasound probe and/or a part of a patient's body where the ultrasound probe is being applied. That is, the video camera is oriented so that it can capture where and how the probe is being positioned on the body of the patient. In addition to this live video feed, ultrasound imagery that is generated by the ultrasound probe and its associated equipment is also transmitted in (near) real time to the device 104. When the video feed and the ultrasound imagery is transmitted to a remote device (e.g., specialist telemedicine device 108/110), a specialist at the remote device can observe the medical procedure and make useful suggestions (e.g., request a repositioning of the probe or a different use of the probe) that can immediately be acted upon by the medical professional who is handling the probe.

It should be noted that the above data streams are generally transmitted in (near) real time. That is, data streams are progressively transmitted while the streams are generated and one or more diagnostic tests are ongoing. When a medical imagery/sensing device detects a change in a physiological condition of the patient, this event is immediately registered with the patient telemedicine device in the form of a change in a transmitted medical image, waveform or data. These images, waveforms or data are also selectively displayed in real time on a display device 210 (step 620).

Put another way, it is common that multiple, different types of data that are displayed on the display device 210 will simultaneously indicate a change that is caused by the same physiological change(s) in the patient. By way of example, consider a situation in which an ultrasound scan of a patient's heart is being performed. At the same time, a heart rate monitor is being used on patient. A video camera takes footage of the patient while they are undergoing a medical examination.

If the patient begins to hyperventilate, this physiological change will impact all of the above sensing devices. That is, a waveform generated by the heart rate monitor will indicate a rise in beats per minute. The video camera footage indicates a tremor in the patient. The ultrasound scan reveals a quickening in the activity of the heart. Data indicating these changes are received simultaneously at the patient telemedicine device 104 and the aforementioned changes are immediately and simultaneously represented at the display device 210 in the form of changes in the heart rate waveform, the video footage and the ultrasound imagery. As will be discussed later in this method 600, the frames of two or more of these data streams will be encoded and synchronized, so that this timing is also conveyed to any remote participants and devices in the telemedicine session.

While the above data is being collected, a medical professional who is handling one of the scanning devices or another participant may wish to send audio messages and commentary to other remote participants in the telemedicine session. For example, a technician who is handling an ultrasonic probe may wish to ask a remote specialist whether the probe is properly being applied to the patient, or to comment on an anomaly he or she noticed in the medical images. In some implementations, the participant speaks into a microphone 208 to create the message. The audio message is then transmitted to and received by the patient telemedicine device 104 (step 608).

As previously discussed in connection with FIGS. 2 and 3, the above data and media is selectively transmitted in (near) real time to remote specialists and other participants (e.g., specialist telemedicine device 108). Upon viewing the images and the diagnostic procedure in real time, a specialist may wish to provide audio commentary or requests, annotate some of the received images, or suggest use cases, reference imagery or other resources. Such collaboration data is transmitted from the specialist telemedicine device(s) 108/110, received, rendered and/or displayed in (near) real time at the patient telemedicine device 104 (step 610).

In various designs, an operator of the patient telemedicine device 104 obtains supplementary data (e.g., use cases, reference imagery, medical records, etc.) from a cloud-based server or database (step 612). Additionally, in various implementations, the operator of the patient telemedicine device 104 annotates or marks any displayed images, waveforms or data (step 614). Any of steps 602, 604, 606, 608, 610, 612 and 614 may be performed using any of the techniques and features previously discussed in connection with FIGS. 2 and 3.

Some designs involve storing any of the above received data for later reference (step 616). Such data can be stored in any suitable storage medium e.g., a flash drive, a hard drive, a connected or remote database, etc. The operator of the patient telemedicine device 104 can provide input to the device, causing the device to obtain and display any stored data.

One or more of the above received data types (e.g., biometric images, biometric data, collaboration data, supplementary data, biometric waveforms, video, etc.) are selectively rendered and displayed in real time at the display device 210 (step 620). At any time, the operator of the patient telemedicine device 104 can configure the device 104 to remove data from the display, to add data to the display, or otherwise arrange the displayed media (step 618). For example, biometric waveforms, patient records, biometric images, video and supplementary data can be presented in separate, resizable and movable windows, as shown in the user interface 400 of FIG. 4.

At almost any time, the operator of the patient telemedicine device can determine data sharing preferences. In some embodiments, the operator of the patient telemedicine device provides input to the device, indicating what kinds of data (e.g., biometric imaging, biometric data, audio, video, collaboration data, supplementary data, etc.) should be shared and what telemedicine devices and professionals should receive the data (step 622). By way of example, an operator could indicate that all biometric imaging, waveform and biometric data received from the medical imaging/sensing devices should be shared with all other participants (e.g., specialist telemedicine devices) in a telemedicine session, but that any annotations and selected medical records only be shown locally at the display device 210.

Generally, the data to be shared is progressively encoded, encrypted and transmitted as it is received or created. In various embodiments, these steps are performed by a data encoding module 800, which is stored at the patient telemedicine device 104.

The data encoding module 800 is any software or hardware that is suitable for encoding and/or encrypting data streams. An example of a data encoding module 800 is illustrated in FIG. 8. The data encoding module 800 is arranged to receive multiple, different types of data streams from medical imaging/sensing devices or other types of devices (e.g., a microphone, a video camera, etc.) and to separately encode and/or process the data streams. In the illustrated embodiment, the module receives audio data, video data, biometric imaging data, biometric data and annotation data, although any of the aforementioned data streams received by the patient telemedicine device 104 may also be processed using the data encoding module.

The data encoding module 800 includes multiple submodules 802a-802e that separately process each data type in parallel. That is, submodules 802a-802e receive and process an audio data stream, video data stream, biometric imaging stream, biometric data and annotation data, respectively. Each submodule includes an authenticator 806, an encoder 808 and an encrypter 810. The authenticator 806 helps ensure that only authorized users are able to direct the encoding process and obtain access to the received data streams. The encoder 808 processes, compresses and/or encodes the associated data stream. The encoder 808 outputs frames obtained from the associated data stream. The encrypter 810 is arranged to at least partially encrypt some or all of the frames. The functionality of these various components will be discussed in greater detail below in the context of method 600 of FIGS. 6 and 7.

Returning to method 600 of FIGS. 6 and 7, the data encoding module 800 separately encodes each type of data. The encoding process involves several operations. Frames are obtained from each associated data stream. In various embodiments, frames are provided by an external device (e.g., a medical imaging/sensing device). In some embodiments, a data stream is received and frames are encoded from the data. A frame is a segment, packet or amount of data of any suitable size. In various embodiments, a frame of a video stream or a biometric imaging stream includes an image, although this is not a requirement. At step 623, each encoder 808 compresses (if appropriate) the frames of its associated data stream. Typically, different data streams will be compressed to different degrees, depending on the use and nature of the data. For example, a video stream of a medical professional's face is not a high priority, does not require exceptionally high resolution and can be easily compressed using a wide variety of compression schemes. On the other hand, an ultrasound image tends to have large amounts of noise, which makes compression somewhat more difficult. Additionally, it is generally desirable that transmitted ultrasound images retain a high resolution to facilitate review and diagnosis. Thus, ultrasound images tend to have lower compression ratios than some types of video. In some embodiments, ultrasound image streams are compressed at a ratio of approximately 1:40 to 1:90, while video is compressed at a ratio of approximately 1:250 to 1:1000, although higher and lower compression levels may also be used for particular applications.

In some embodiments, the level and type of compression for some data streams (e.g., biometric imaging streams, such as ultrasound images) is determined dynamically. That is, feedback is received from one or more quality of service agents. The compression scheme used on the data stream is based at least in part on this feedback. Various techniques and arrangements relating to such compression schemes are described in U.S. patent application Ser. No. 14/291,567, entitled “Dynamic Adjustment of Image Compression for High Resolution Live Medical Image Sharing”, filed May 30, 2014, which is incorporated herein in its entirety for all purposes. Any method, component, system, operation or arrangement described in this application may be used to compress a suitable biometric imaging stream or other data stream in step 623.

At step 624, the encoder 808 adds a timestamp to each frame. In various implementations, this timestamp is inserted into a header of the frame. Generally, the timestamp represents an approximate time at which the frame was processed, generated or received at a local device (e.g., the patient telemedicine device 104.) The timestamp can involve any time, value or code that helps indicate time, timing or an order in which frames should be rendered. The timestamp is used to help ensure that frames of particular data streams that are reconstructed at a remote device are properly synchronized and coordinated, as will be discussed in greater detail below. The timestamp may be derived from any suitable source. In some implementations in which the two synchronized streams are received and encoded at the same computing device (e.g., the patient telemedicine device 104), the timestamp can be based on a timer, clock or CPU clock of the computing device. Alternatively, the timestamp can be based on a time received through the network e.g., using the Network Time Protocol (NTP), from a NTP time server or other type of time server, etc. In some applications, the timestamp is based on time data in a GPS signal received from a GPS satellite. These network- or GPS-based approaches work well when synchronization is required between two data streams that originated from different locations or computers in a network.

At step 636 of FIG. 7, the encrypter 810 in each submodule receives the associated frames from the encoder and at least partially encrypts them. Any known encryption technology may be used. In various embodiments, frames from different types of data streams are separately encrypted using different encryption techniques, depending on the characteristics of the data.

Various encryption operations are illustrated in FIGS. 9A-9D. FIGS. 9A-9D illustrate frames 900. Each frame includes a header 902 and media data/payload 904. The media payload 904 contains a particular media type (e.g., audio, video, biometric imagery, biometric data, etc.) The header 902 includes metadata relating to the media in the media payload 904 of the frame. In various embodiments, for example, the header 902 indicates characteristics of the media payload 904 and/or includes information on how to access, process and/or render the media data. The shaded regions represent parts of the frames that are encrypted, while the white areas of the frames are unencrypted parts. Thus, in FIG. 9A, only the entire header 902 is encrypted. In FIG. 9B, only a portion but not all of the header is encrypted. In FIG. 9C, a portion of the header 902 and a portion of the payload 904 of the frame is encrypted. In FIG. 9D, the entire frame or almost the entire frame is encrypted. In some embodiments, only a particular type of frame is encrypted while other types of frames are not encrypted.

The advantage in encrypting only a portion of a frame or particular types of frames is that it substantially reduces overhead. In various applications, selected portions of each frame are encrypted (and the other portions are left unencrypted) such that an interceptor of the frame would not be able to make sense of the contents of the frame without decrypting those selected portions. The encrypted portion can thus vary depending on the data type and packet structure. That is, video frames may be encrypted in a different manner than biometric imaging frames, etc.

The type of data that is encrypted in each frame can vary, depending on the type of data and the needs of a particular application. By way of example, for multichannel data streams (e.g., from a medical sensing device, such as an electrocardiogram device, electroencephalography device, a pulse oximeter device, a thermal/temperature sensor, a blood pressure testing device, a glucose level testing device, a pulmonary function testing device, etc.), the encrypted portion(s) of a header may indicate a channel ID and/or sampling rate. For biometric imaging streams or video, the encrypted portion(s) of a header of each frame can indicate a number of macroblocks in a portion of the frame, a quantization table, and/or a DC coefficient of each of the macroblocks. In other approaches, a particular type of frame (e.g., an I-, P- or B-frame for video streams) is encrypted and another type of frame is left unencrypted. In still other embodiments, the media payload of a biometric imaging or video frame contains an image, which is divided into multiple slices. Each slice has a header, which is encrypted, although the slices themselves are not.

Returning to FIG. 7, after the encryption operations, the data stream encoding module breaks the frames from the various data types down into packet sequences, multiplexes the packet sequences and transmits them (step 638) to a remote device (e.g., to a telemedicine device 108/110). The transmission may be performed using any suitable network protocol, such as UDP or TCP/IP. If a server 116 is available in the multi-site data sharing platform, all traffic may pass first through one or more servers, and then be broadcasted to any participating specialist telemedicine devices from the server(s). Alternatively, the packet sequences may be sent to a single or multiple telemedicine devices directly. In various embodiments, when frames with a particular type of encryption are to be sent, an encrypted message is transmitted to the server or the remote devices, which indicate what portions of each frame are encrypted. This allows an appropriately authorized device at the receiving end to access the frames that follow the encrypted message.

Once the packet sequences are received at the remote device, the remote device reconstructs frames of the various data streams based on the packet sequences (step 640). For example, the biometric imaging packet sequence is used to reconstruct the frames of the biometric imaging stream, the video packet sequence is used to reconstruct the frames of the video stream, and so on. Each reconstructed frame includes a timestamp, as noted in step 624.

For various applications, it is desirable to synchronize frames from two or more data streams. Steps 644, 646 and 648 pertain to the synchronization of different combinations of data streams for different applications. It should be appreciated, however, that any suitable group of different data streams may be synchronized.

Generally, the synchronization of two data streams involves rendering and displaying the frames of the two data streams in order based on their associated timestamps. Put another way, the frames are rendered and displayed at the remote device in the order that they were generated or originally received at the patient telemedicine device 104. If a frame of one data stream was received at the patient telemedicine device at the same time as the frame of another data stream, if the frames are properly synchronized, the two frames will be rendered and displayed simultaneously at the remote device as well, since the two frames should have similar or identical timestamps.

Synchronization is desirable for a variety of applications and data stream combinations. Step 644, for example, pertains to the optional synchronization of frames of a video stream and a biometric imaging stream. To illustrate the value of such synchronization, consider an example in which a patient telemedicine medical device 104 receives a video stream and a biometric imaging stream. The video stream pertains to live video footage of a medical professional handling an ultrasound probe or another medical imaging/sensing device. The biometric imaging stream is received from the medical imaging/sensing device that the professional is handling. That is, the actions of the professional directly and immediately affect what is shown in the biometric imaging stream.

In this kind of situation, it would be desirable if a remote specialist participating in a telemedicine session could watch the professional's use of the device and the resulting biometric imagery in real time and provide suggestions on how to position or use the device. This works well only if the remote specialist perceives little or no delay between the use of the device and the resulting biometric imagery. The rendering and display of the frames of the data streams in order based on the associated timestamps helps ensure such synchronization.

Step 646 pertains to the optional synchronization of frames of biometric waveform and biometric imaging streams. Such synchronization is useful in applications where different diagnostic tests are being applied to the same patient and provide different types of information about the same physiological changes. Consider an example in which an ultrasound probe is being applied to a patient and is generating an ultrasound image of the patient's heart. Additionally, a heart rate monitor is applied to the patient, which is continuously monitoring heart activity and outputting a waveform that tracks the heart activity. Particular changes in the activity of the heart (e.g., a sudden burst of palpitations or a seizure) would simultaneously register in both the waveform and the ultrasound imagery that is being received at the patient telemedicine device in (near) real time. The synchronization of the frames of the biometric waveform and biometric imaging streams allows telemedicine participants to view the waveform and images in (near) real time with the correct timing and order.

Step 648 pertains to the optional synchronization of frames of annotation data and the biometric imaging stream. As previously discussed, in various embodiments any operator of a specialist telemedicine device 108/110 or the patient telemedicine device 104 can annotate frames of a displayed biometric image (e.g., an ultrasound image). In some cases, the annotation takes the form of a circle, line, an underline, a highlight or any other suitable mark. Generally, it is desirable that the association between an annotation and the underlying image or frame be preserved, even as the annotation data and imaging data is streamed to different devices. That is, when the annotation data is transmitted to a remote device and re-rendered, it should be rendered simultaneously with the imaging frames to which the annotation was applied.

Such synchronization can be achieved as follows. Each frame of annotation data represents or includes an annotation that was applied to a particular frame of a biometric imaging stream. In step 624, very similar or the same timestamps are added to the annotation frame and the biometric imaging frame that the annotation was applied to. As previously discussed, the frames of the annotation data and biometric imaging stream are used to form packets. The packets are then transmitted and received at a remote device. At the remote device, the packets are used to reconstruct the frames of the annotation data and biometric imaging streams (step 640), which include the aforementioned timestamps. The reconstructed frames of the annotation data and biometric imaging streams are rendered and displayed in the order of their associated timestamps (i.e., since their timestamps are very similar or the same, the annotation and biometric imaging frames are rendered and displayed (approximately) simultaneously.) As a result, an annotation that was applied to a frame of a biometric imaging stream at a local device can be displayed together with the same frame of the biometric imaging stream at a remote device. That is, at the remote device there should be no or minimal delay between the display of the annotation and the frame of biometric imaging to which the annotation was applied.

The aforementioned steps in FIGS. 6-7 are illustrated in a particular order, but it should be appreciated that in various implementations many of the steps are performed simultaneously (e.g., steps 602, 604, 606, 608, 610, 612 and 614 can occur simultaneously) or may be performed in a different order. Although the method 600 is generally performed by a patient telemedicine device 104, many of the steps (e.g., steps 608-638) can also by performed by any specialist telemedicine device 108/110. Although each step involves particular types of operations, these operations may be modified as appropriate using any suitable techniques known to persons of ordinary skill in the art.

Any of the methods (e.g., methods 600 and 700 of FIGS. 6 and 7), processes, actions and techniques (e.g., operations performed by the patient telemedicine device 104 and/or the specialist telemedicine device 108 in connection with any of the figures) described herein may be stored in the form of executable computer code or instructions in a tangible computer readable medium (e.g., in a hard drive, a flash drive, any suitable type of computer memory, etc.) In some embodiments, the computer code or instructions is stored in at least one memory of a device (e.g., a patient telemedicine device 104 or a specialist telemedicine device 108/110.) The device also includes at least one processor. The computer code or instructions, when executed by the at least one processor, causes the device to perform any of the operations or methods described herein.

Although only a few embodiments of the invention have been described in detail, it should be appreciated that the invention may be implemented in many other forms without departing from the spirit or scope of the invention. For example, there are several references in the application to a “local device” communicating with a “remote device.” It should be appreciated that the local device can refer to any device in the multi-site data sharing platform 100 (e.g., a patient telemedicine device 104, a specialist telemedicine device 108/110). The remote device refers to any other device that is connected with the local device through the cloud and/or a network and that is also in the multi-site data sharing platform 100 (e.g., a patient telemedicine device 104, a specialist telemedicine device 108/110, etc.). Various block diagrams have been presented in this application. However, it should be appreciated that the features and operations of one component in the diagram may be transferred to another component. Additionally, each component may be divided into multiple separate components or and/or merged with one or more other components. Some figures, such as FIGS. 2 and 3, include multiple components, inputs and data streams. It should be noted that in some implementations, fewer or more components, inputs and data streams may be involved. For example, FIG. 2 illustrates a patient telemedicine device 104 that receives simultaneous data streams from a probe 106b, a biometric waveform data source 106c and a biometric imaging source 106d. However, this application also contemplates embodiments in which, for example, biometric data is received from the probe and probe platform (e.g., an ultrasound probe), video is received from the video camera (e.g., video footage of the application and use of the probe on the body of a patient) and no biometric waveform data source and/or biometric imaging source is used. In this application, there are references to a “telemedicine device” (e.g., a specialist telemedicine device, a patient telemedicine device, etc.) In some of the figures, the telemedicine device is depicted as having particular functions. For example, the patient telemedicine device is depicted as receiving data from multiple components and sources. However, it should be appreciated that the patient telemedicine device is not necessarily a single structure and in various embodiments is a system that includes multiple connected components that provide additional features or functionality. For example, the patient telemedicine device can incorporate or include additional adapters, connectors, modules, devices and/or any component described in the telemedicine system 102. In some embodiments, the patient telemedicine device incorporates an image acquisition device 202, a microphone 208, a video camera 106a, a diagnostic device, etc.) Therefore, the present embodiments should be considered illustrative and not restrictive and the invention is not to be limited to the details given herein.

What is claimed is:

1. A method for sharing medical data in a telemedicine session comprising:
receiving a biometric imaging stream in real time from a medical scanning device;
receiving a data stream in real time, the data stream being one selected from the group consisting of a video stream received from a video camera and a biometric data stream that is received from a medical sensing device wherein the biometric imaging stream and the data stream are received simultaneously;
obtaining a plurality of biometric imaging frames from the biometric imaging stream as the biometric imaging stream is being received;
obtaining a plurality of data frames from the data stream as the data stream is being received;
inserting timestamps into the biometric imaging and data frames so that they can be rendered synchronously in real time on a remote device; and
transmitting the biometric imaging and data frames in real time to the remote device while the biometric imaging and data streams are being received so that the biometric imaging and data frames can be rendered in substantially real time at the remote device.

2. A method as recited in claim 1 wherein:
the biometric imaging stream is received from a medical scanning device that is being applied to a patient;
the video stream is received from a video camera that is directed at a part of the body of the patient; and
the biometric data stream is received from a medical sensing device that is being used on the patient.

3. A method as recited in claim 1 further comprising:
receiving the biometric imaging and data frames at the remote device; and
rendering the biometric imaging and data frames in an order based on the timestamps.

4. A method as recited in claim 1, the method further comprising:
partially encrypting the biometric imaging and data frames such that at least a portion of one or more of the frames is not encrypted.

5. A method as recited in claim 4 further comprising:
the encrypting involves at least one selected from the group consisting of (1) encrypting a particular type of frame and not encrypting another type of frame; (2) encrypting a part of a frame and leaving other parts of the frame; (3) encrypting at least a portion of a header of a frame; (4) encrypting a portion of a header of a frame and a portion of media payload in the frame while leaving other parts of the frame unencrypted; and (5) encrypting only a header and not a media payload of the frame.

6. A method as recited in claim 4 further comprising:
transmitting a message to the remote device indicating which part of each frame is encrypted.

7. A method as recited in claim 1 further comprising:
compressing the biometric imaging stream at a first level of compression;
compressing the video stream at a second level of compression wherein the first level of compression is substantially less than the second level of compression.

8. A method as recited in claim 1 wherein:
the biometric imaging stream is generated by at least one selected from the group consisting of an ultrasound device, an magnetic resonance imaging (MRI) device, a computed tomography (CT) device and an X-ray device.

9. A method as recited in claim 1 further comprising:
receiving from a user an annotation on a particular biometric imaging frame;
generating an annotation frame from the annotation;
adding an annotation timestamp to the annotation frame and the biometric imaging frame so that when the annotation and biometric imaging frames are reconstructed and rendered at the remote device, the annotation and the biometric imaging frames are synchronized; and
transmitting the annotation frame and the biometric imaging frame to the remote device.

10. A method as recited in claim 1 wherein:
the biometric imaging stream is an ultrasound image stream that is generated using an ultrasound probe and that is received in real time; and
the data stream is a video stream that is received in real time from a video camera wherein the video camera is directed at a body of a patient such that the video stream indicates where the ultrasound probe is being positioned on the body of the patient and the biometric imaging stream simultaneously indicates an ultrasound scan of the portion of the body where the probe is positioned.

11. A method as recited in claim 1 wherein:
the receiving of the biometric imaging stream and the obtaining of the plurality of biometric imaging frames are performed at a first telemedicine device that is at a first location on a network;
the receiving of the data stream and the obtaining of the plurality of data frames are performed at a separate second telemedicine device that is at a second location on the network that is different from the first location; and
the method further comprises:
inserting a first timestamp into one of the biometric imaging frames at the first telemedicine device wherein the first timestamp is obtained at the first telemedicine device using a timing source selected from the group consisting of an NTP server and a GPS satellite; and
inserting a second timestamp into one of the data frames at the second telemedicine device wherein the second timestamp is obtained at the second telemedicine device using a timing source selected from the group consisting of an NTP server and a GPS satellite; and
the one of the biometric imaging frames is obtained at the first telemedicine device at approximately the same time as the one of the data frames is obtained at the second telemedicine device, thereby causing the first timestamp to be approximately the same as the second timestamp and helping to ensure that the ones of the biometric imaging and data frames are rendered approximately simultaneously at the remote device based on the first and second timestamps.

12. A method for collaborating in a telemedicine session comprising:
receiving biometric imaging frames in real time from a transmitting device over a network wherein the biometric imaging frames are generated at least in part by a medical scanning device;
receiving data frames from the transmitting device over the network, the data frames being one selected from the group consisting of video frames that are generated at least in part by a video camera and biometric data frames that are generated at least in part by a medical sensing device wherein the biometric imaging frames and the data frames are received simultaneously as they are being generated and transmitted;
obtaining timestamps from the biometric imaging and data frames;
synchronizing the biometric imaging and data frames based on the timestamps; and
rendering the synchronized biometric imaging and data frames synchronously in real time as the biometric imaging and video frames are received from the transmitting device over the network.

13. A method as recited in claim 12 wherein:
the biometric imaging frames are generated at least in part by a medical scanning device that is being applied to a patient;
the video frames are generated at least in part by a video camera that is directed at a part of the body of the patient; and
the biometric data frames are generated at least in part by a medical sensing device that is being used on the patient.

14. A method as recited in claim 12 wherein the synchronized frames are rendered in order based on the timestamps.

15. A method as recited in claim 12 further comprising:
receiving from a user an annotation on a particular biometric imaging frame;
generating an annotation frame from the annotation;
associating the annotation frame and the biometric imaging frame with an annotation timestamp;
inserting the annotation timestamp into the annotation frame and the biometric imaging frame so that when the biometric imaging frame and the annotation frame are rendered at a remote device, the annotation and the biometric imaging frame are synchronized; and
transmitting the biometric imaging and annotation frames to the remote device.

16. A method as recited in claim 12 wherein:
the biometric imaging device is at least one selected from the group consisting of an ultrasound device, a magnetic resonance imaging (MRI) device, a computed tomography (CT) device and an X-ray device; and
the medical sensing device is at least one selected from the group consisting of an electrocardiogram device, electroencephalography device, a pulse oximeter device, a thermal/temperature sensor, a blood pressure testing device, a glucose level testing device, and a pulmonary function testing device.

17. A method for selectively encrypting medical data, the method comprising:
receiving a data stream from a medical imaging/sensing device;
obtaining a plurality of data frames from the data stream;
selectively encrypting a portion of the data frames and leaving another portion of the data frames unencrypted; and
transmitting the data frames to a remote device as the data stream is being received.

18. A method as recited in claim 17 wherein:
the selective encryption involves at least one selected from the group consisting of: (1) encrypting a particular type of frame and not encrypting another type of frame; (2) encrypting a part of each frame and leaving other parts of the frame unencrypted; (3) encrypting at least a portion of a header of each frame; (4) encrypting a portion of a header of each frame and a portion of media payload of the frame while leaving other parts of the frame unencrypted; and (5) encrypting only a header of each frame and not a media payload of the frame.

19. A method as recited in claim 17 wherein:
the data stream involves multichannel data obtained from a medical sensing device;
the medical sensing device is at least one selected from the group consisting of an electrocardiogram device, electroencephalography device, a pulse oximeter device, a thermal/temperature sensor, a blood pressure testing device, a glucose level testing device, and a pulmonary function testing device; and
the selective encryption involves encrypting control information in a header of each frame and leaving other parts of the frame unencrypted and wherein the control information indicates at least one selected from the group consisting of channel ID and sampling speed.

20. A method as recited in claim 17 wherein:
the data stream is at least one selected from the group consisting of a video stream received from a video camera and a biometric imaging stream received from a medical scanning device; and
the selective encryption involves encrypting control information in a header of each frame and leaving other parts of the frame unencrypted and wherein the control information indicates at least one selected from the group consisting of (1) number of macroblocks in a portion of the frame; (2) a quantization table; and (3) DC coefficient of each of a plurality of macroblocks in the frame.

21. A telemedicine device, comprising:
at least one processor;
at least one memory that stores computer readable instructions, which when executed by the at least one processor cause the telemedicine device to:
receive a biometric imaging stream in real time from a medical scanning device;
receive a data stream in real time, the data stream being one selected from the group consisting of a video stream received from a video camera and a biometric data stream that is received from a medical sensing device wherein the biometric imaging stream and the data stream are received simultaneously;
obtain a plurality of biometric imaging frames from the biometric imaging stream as the biometric imaging stream is being received;
obtain a plurality of data frames from the data stream as the data stream is being received;
insert timestamps into the biometric imaging and data frames so that they can be rendered synchronously in real time on a remote device; and
transmit the biometric imaging and data frames in real time to the remote device while the biometric imaging and data streams are being received so that the biometric imaging and data frames can be rendered in substantially real time at the remote device.
